(19) United States
(12) Patent Application Publication
(10) Pub. No.: US 2024/0327940 A1
(43) Pub. Date: Oct. 3, 2024

(54) A method and associated system to characterize an alloy's hardenability for finite element analysis

(71) Applicants: Joseph POWEL, Uniontown, OH (US); William STUEHR, North Royalton, OH (US); Integrated Heat Treating Solutions, LLC, Akron, OH (US)

(72) Inventors: Joseph A Powell, Uniontown, OH (US); William Stuehr, North Royalton, OH (US)

(73) Assignee: Integrated Heat Treating Solutions, LLC, Akron, OH (US)

(21) Appl. No.: 18/579,332

(22) PCT Filed: Jul. 14, 2022
(86) PCT No.: PCT/US2022/037167

§ 371 Date: Jan. 13, 2024

Related U.S. Application Data
(60) Provisional application No. 63/222,029, filed on Jul. 15, 2021.

(51) Int. Cl. C21D 1/55

(52) U.S. Cl. CPC C21D 1/55

(57) ABSTRACT

A system and apparatus for conducting a modified Jominy end-quench test of a metal composition having an Austenitizing temperature and a critical cooling rate may comprise a bar, comprising the metal composition, said bar having a hemispherical tip and a bar length. The system may also comprise a bar receptacle. The system may also comprise a quenchant. Additionally, the system may also comprise a quenchant discharge apparatus as a quenchant nozzle. In the system a portion of the hemispherical tip of the bar passes through a portion of the bar receptacle, exposing it to a quenchant exiting the quenchant nozzle with a gap existing between the quenchant nozzle and the hemispherical tip of the bar. This is useful in characterizing an alloy's hardenability and residual stresses for finite element analysis and part design.

### CROSS REFERENCES AND PRIORITIES

This application claims priority from U.S. Provisional Patent Application 63/222,029 filed on 15 Jul. 2021, the teachings of which are incorporated by reference herein in their entirety.

### BACKGROUND

When hot Martensitic steel alloys are quenched during heat treating, the area in contact with the quenchant should rapidly cool to the Martensite start temperature creating a hard shell. The surface of the material equilibrates with the temperature of the quenchant being used to quench. The layers below the outer shell of the part—i.e. the inner area of the metal—does not cool as rapidly as the outer shell. In some cases, the inside cools too slowly to transform primarily to Martensite, resulting in a different internal structure than the outer shell. This results in a harder, tough outer shell and a softer, more durable core.

A hardenability test measures the depth to which a sample of a specific material is hardened after putting it through a heat treatment process that includes a quench with a rapid cooling rate. Depending on the amount and type of alloying elements in the grade of Martensitic material, the objective of hardening the material can be achieved through a variety of methods—e.g. air hardening steels; water hardening steels or oil hardening steels.

One such test for determining hardenability of a metal or metal alloy is a Jominy end-quench test. In a traditional Jominy end-quench test, a test sample of a particular metal or metal alloy is heated to at least the Austenitizing temperature of the metal or metal alloy. The sample is then transferred to a test fixture which quenches the sample by spraying a controlled flow of water (with or without dissolved rust preventatives) onto one end of the sample.

Traditional Jominy tests are conducted according to ASTM Jominy End-Quench Test No. A 255-20a, last updated 16 Nov. 2020, which is incorporated by reference herein in its entirety. However, the traditional Jominy end-quench test has many defects which may impact the ability to achieve reliable and repeatable results. These defects include inconsistencies in the area of the sample exposed to the quenchant during quenching. Additionally, since the quenchant is discharged, there is the potential for variation in the angle of impact onto the test sample. At the same time, the time spent transferring the sample from the Austenitizing furnace to the test fixture results in a degree of unintended air quenching. Heat conduction between the sample and the test fixture or the tongs used to remove the sample from the furnace are additional sources of variation. Additionally, traditional Jominy end-quench tests are incapable of determining the ultimate hardenability of a specific metal or metal alloy because of the relatively slow cooling rate from the low pressure contact between the quenchant and the end-point of the sample material that enables the liquid quenchant to boil creating a steam blanket that insulates the sample and reduces the cooling rate.

The traditional Jominy end-quench cooling rate is also too slow to enable the creation of the transient crystal structures required to create compressive surface stresses—sometimes referred to by practitioners as “current” compressive surface stresses—at the end of the sample bar. Without the instantaneous creation of these “current” compressive surface stresses at the initiation of the quench on the part shell, the traditional Jominy test cannot predict the level of residual compressive surface stresses—if any exist at all—that are available from a given hardenability of alloy for modeling a given part mass and geometry. In essence the standard Jominy test only yields tensile stresses.

The need exists, therefore, for an improved system and method for conducting a Jominy end-quench test that allows one to determine the current and ultimate achievable compressive surface stresses and hardenability.

### SUMMARY

A system for conducting a modified Jominy end-quench test of a metal composition having an Austenitizing temperature, and a critical cooling rate is disclosed. The system comprises a bar comprising the metal composition, a bar receptacle, a quenchant, and a quenchant discharge apparatus. Said bar comprising the metal composition also comprises a hemispherical tip and a bar length. The bar receptacle comprises a rigid collar with a top surface, a bottom surface opposite the top surface, a through hole from the top surface through the bottom surface, and a brace. The quenchant discharge apparatus comprises a quenchant nozzle. A portion of the hemispherical tip of the bar passes through the through hole to form an exposed portion of the hemispherical tip of the bar located between the bar receptacle and the quenchant nozzle, with a gap existing between the quenchant nozzle and the hemispherical tip.

In some embodiments the quenchant nozzle may be configured to discharge the quenchant to cool at least the exposed portion of the hemispherical tip of the bar at a cooling rate equal to or greater than the critical cooling rate of the metal composition of the bar.

In some embodiments, the system may comprise a shield attached to the bar receptacle. In some embodiments, the system may comprise a reinforcement, connecting the bar receptacle and the shield.

In some embodiments, the gap between the quenchant nozzle and the hemispherical tip may be between 5 millimeter and 5 centimeters. In other embodiments, the gap between the quenchant nozzle and the hemispherical tip may be between 5 millimeter and 2.5 centimeters.

In some embodiments, the system may be configured to cool the exposed portion of the hemispherical tip without film boiling.

A method to determine at least one property of the metal composition having an Austenitizing temperature and a critical cooling rate is also disclosed. In the method at least a surface of the bar at the exposed portion of the hemispherical tip of the bar is heated to a temperature greater than or equal to the Austenitizing temperature. A portion of the hemispherical tip of the bar is then passed through the through hole of the bar receptacle. All but the exposed portion of the hemispherical tip of the bar is shielded to keep the quenchant from contacting any surface of the bar other than the exposed portion of the hemispherical tip. The exposed portion of the hemispherical tip of the bar is then contacted with quenchant. The bar is then analyzed for the at least one property.

In some embodiments the exposed portion of the hemispherical tip of the bar is contacted with the quenchant at a pressure greater than a boiling pressure of the quenchant at the through temperature of the bar and at a quenchant flow renewal rate for a time.

In some embodiments, the property the is analyzed for is selected from a group consisting of ultimate hardenability, compressive stresses, tensile stresses, crystal structure, grain size, hardness, and combinations thereof.

In some embodiments, the bar of the metal composition may be heated to at least the Austenitizing temperature using induction heating.

In some embodiments, the hemispherical tip is cooled at a cooling rate equal to or greater than the critical cooling rate of the metal composition.

In some embodiments, the quenchant exiting the quenchant nozzle may be located between 5 millimeter and 5 centimeters from the hemispherical tip. In other embodiments, the quenchant exiting the quenchant nozzle may be located between 5 millimeters and 2.5 centimeters from the hemispherical tip.

In some embodiments, the method may further comprise analyzing the compressive surface stresses along the length of the bar.

In some embodiments, the method may further comprise determining the ultimate hardenability of the metal composition of the bar through measuring hardness at a plurality of depths.

### BRIEF DESCRIPTION OF FIGURES

FIG. 1 is a top view of an embodiment of the invented system for conducting a modified Jominy end-quench test.

FIG. 2A is a side cross section view of an embodiment of the invented apparatus for conducting a modified Jominy end-quench test.

FIG. 2B is a side cross section view of an embodiment of the invented system, inclusive of the modified Jominy bar, for conducting a modified Jominy end quench test.

FIG. 3 are the cross sectional profiles of two modified Jominy bars with a hemispherical tip and a conical tip, respectively.

FIG. 4 is a cross section profile of a quenched bar using a single quenchant spray at the very tip of the hemispherical end.

FIG. 5 is an embodiment of a nozzle and modified Jominy bar of the invented system.

FIG. 6 is an exemplary TTT diagram demonstrating the derivation of the critical cooling rate for the measured material.

### DETAILED DESCRIPTION

Disclosed herein is a process for testing of a metal alloy having an Austenitizing temperature and a critical cooling rate and gathering data that can be developed from analyzing an alloy of Martensitic steel after quenching. Specifically, an improved system for conducting a modified Jominy end-quench test is disclosed. Also disclosed herein is a method for determining one or more properties of a metal composition after Austenitizing heating using uniform induction heating and consistent transfer time before quench cooling.

The following is a list of numeral referents depicting the elements of the invention.
10 refers to the system, which includes the bar.
20 refers to the apparatus which is the system without the bar.
100 refers to the modified Jominy bar.
110 refers to the hemispherical tip of the quench end of an exemplary modified Jominy bar.
120 refers to the modified Jominy bar length which is along the height of the cylinder shape of the modified Jominy bar.
130 refers to the conical tip of the quench end of an exemplary modified Jominy bar.
140 refers to the locking end of the modified Jominy bar which is opposite the quench end.
200 refers to the bar receptacle.
210 refers a rigid collar of the bar receptacle.
212 refers to a through hole passing from a top surface of the rigid collar to the bottom surface of the rigid collar.
214 refers to the top surface of the rigid collar.
216 refers to the bottom surface of the rigid collar.
220 refers to a brace which extends from the rigid collar to the frame.
230 refers to the lock plate.
240 refers to the lock mechanism of the apparatus with the locked position in bold and the unlocked, open position in hashes.
250 refers to the frame of the apparatus.
260 refers to the guide plate of apparatus.
265 refers to the through hole in the guide plate.
300 refers to the quenchant discharge apparatus.
310 refers to the quenchant nozzle.
315 refers to a filter in the quenchant discharge apparatus.
320 refers to the fluid connection, or conduit, connecting the quenchant discharge apparatus with the quenchant source.
330 refers to a valve.
400 refers the a shield.
500 refers to the single point of impact of a very narrow quench spray.
510 refers to the plume formed behind the single point of impact.
700 refers to the quenchant.

As used in this specification and in the claims, the term quenchant is a liquid (or air or an inert gas quenchant, e.g., nitrogen, if testing the hardenability of a high alloy “air hardening” alloy of material). The quenchant uniformly draws heat from the hemispherical end of the hot metal part to cool the part by conduction up its axis.

Additionally, as used in this specification and in the claims, the term bar can be made of a metal composition regardless of exterior shape used for sample purposes. This includes but is not limited to metal compositions with square, rectangle, or circular exteriors, commonly referred to as a bar, rod, shaft, beam, or other commonly utilized terms. The preferred bar shape is a straight, round, circular rod, i.e. a cylinder, with a non-flat or non-square, tip at one end. A flat, or square, tip is one which is perpendicular to the cylinder's height. The preferred tip is symmetrical about the cylinder's longitudinal axis with the preferred tip being hemispherical or conical, with the hemispherical tip most preferred.

As well, as used in the specification and in the claims, the term quenchant flow rate refers to the rate at which the quenchant is discharged. This is in contrast to the term quenchant flow renewal rate used to refer to the rate at which quenchant is renewed at a specific point at which the quenchant initially contacts the bar, preferably the hemispherical tip, more preferably the end of the hemispherical tip closest to the quenchant nozzle.

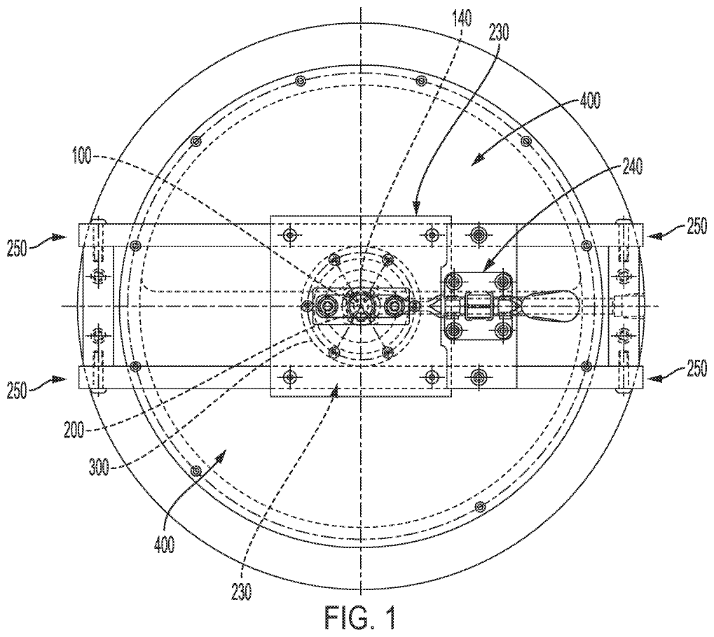

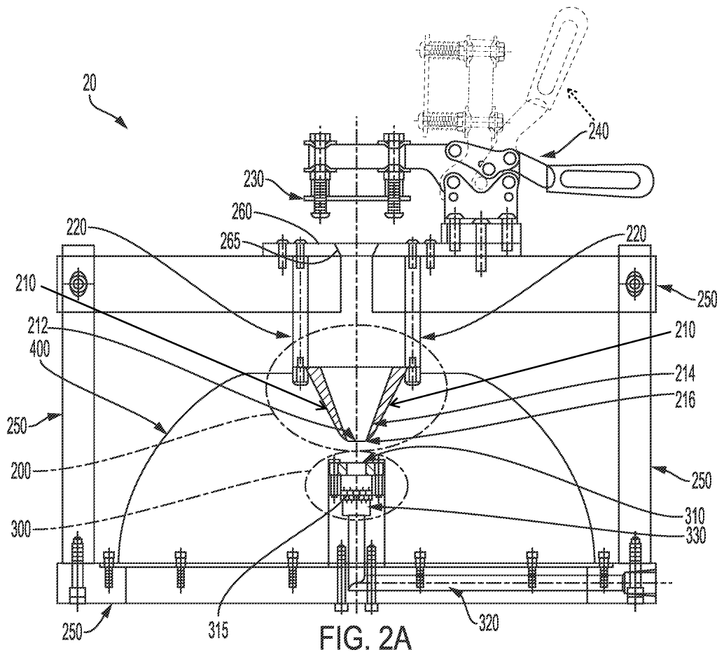

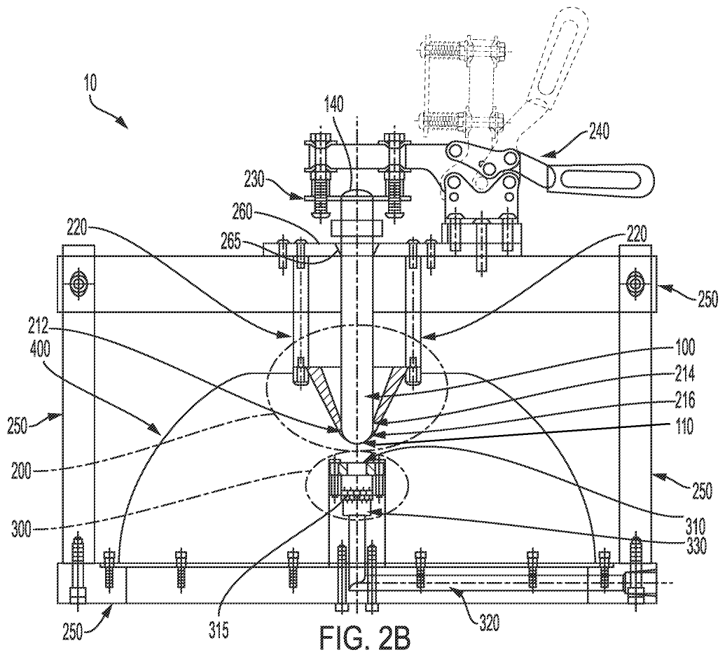

FIG. 1 shows a top view of an embodiment of the invented system for conducting a Jominy end-quench test. As shown in FIGS. 1, 2A, and 2B, the system (10) comprises a bar (100) and apparatus (20), which has a bar receptacle (200), a lock mechanism (300) within a frame (250) and a quenchant discharge apparatus (300).

As shown in FIGS. 1, 2A, and 2B the bar receptacle (200) may comprise a rigid collar (210) comprising a through hole (212) passing from a top surface of the rigid collar (214) through a bottom surface of the rigid collar (216). As shown in FIG. 2A and FIG. 2B, the bar receptacle is conical to better facilitate placing the bar in the same location each time.

The rigid collar will preferably be comprised of a material and structure having a thermal conductivity less than or equal to 25 W/(mK) with less than or equal to 20 W/(mK) being preferred and less than or equal to 15 W/(mK) being most preferred. In some embodiments the bar receptacle may further comprise a brace (220) extending from opposing sides of the rigid collar. The brace may further connect to a shield (400), as shown in FIGS. 1, 2A, and 2B. The system may comprise one or more reinforcements, which are the screws set into the brace (220), connecting the bar receptacle and the shield

The collar is essentially a cone with the tip cut off through which a portion of the bar extends from the tip. The collar and bar are configured so that the quenchant striking the bar only strikes that part of the bar extending from the collar.

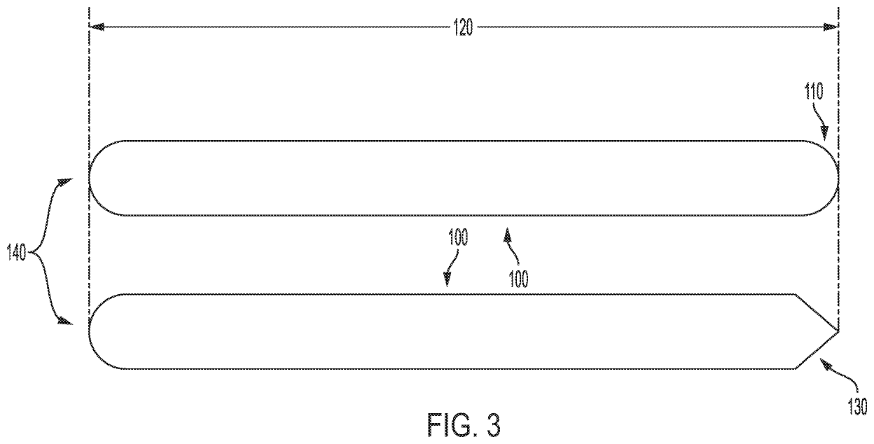

The purpose of the apparatus is to have the quenchant only strike the exposed portion of the bar and no place else. As shown in FIG. 2B, the shield, which is attached to the collar prevents the quenchant from contacting any portion of the bar not extending through the collar. The shield is a bowl shape so that the quenchant striking the bowl is pushed along the edge and down outside the apparatus. FIG. 2A shows a side cross section view of the apparatus (20). FIG. 2B shows the system (10) with the bar. As shown in FIG. 3 the bar (100) will have a bar length (120). It is preferred that the bar have a cylindrical straight round rod profile. At least one end of the bar has a hemispherical or substantially hemispherical tip (110) having a diameter that is the same or substantially the same as the diameter of the cylindrical profile of the bar. The bar will be comprised of a metal composition which is a material of a particular metal or metal alloy to be tested. When the bar is of a metal alloy it may be comprised of primarily iron and a number of different elements commonly known in metal alloys-including, but not limited to carbon, graphite, nickel, manganese, chromium, molybdenum, vanadium, silicon, and boron. Non-limiting examples of a metal or metal alloy include 8620 Steel and ductile iron. Said metal or metal alloy will have a known Austenitizing temperature which will vary with the type of metal or metal alloy being utilized. Some metals or metal alloys may also have a known Martensite start and finish temperature and/or a known bainite formation temperature range.

In conducting the modified Jominy end-quench test, at least a surface of the bar (100) is heated at least at the exposed portion of the hemispherical tip of the bar to a temperature which is at least the Austenitizing temperature of the metal or metal alloy of the bar's composition. Preferably the bar is through heated.

Although a number of heating methods-including uniform and nonuniform heating methods-which are known in the art may be used, the preferred heating method comprises uniform induction heating. The metal bar (100) is then quickly-preferably within no more than 5 seconds with no more than 3 seconds being more preferred and no more than 1 second being most preferred-lowered to rest inside the bar receptacle (200). At least a portion of the hemispherical tip (110) comprising the tangential point of the hemisphere extends through the through hole (212) with the hemispherical tip within the bar receptacle such that a portion of the hemispherical tip is directly exposed to quenchant discharged from the quenchant nozzle (310). All but the exposed portion of the hemispherical tip of the bar is shielded to prevent the quenchant from contacting any surface of the bar other than the exposed portion of the hemispherical tip of the bar. The quenchant from the quenchant nozzle will then only contact the exposed portion of the hemispherical bar tip and not the remainder of the bar.

As shown in FIGS. 2A and 2B, the bar preferably passes through the guide plate hole (265) of guide plate (260). The guide plate hole is configured so that the bar passes through the guide plate hole and the tip of the bar passes through the through hole of the collar, but the bar does not pass through the through hole. That is the size of the through hole in the collar is smaller than the bar. Preferably the through hole in the collar is configured so that the collar is holding the bar at the point where the non-flat tip (conical or hemispherical) begins.

Lock mechanism (240) is used to keep the bar in place and aligned with the collar. As shown in FIGS. 2A and 2B, the lock mechanism is first in the open position indicated by the hashed FIG. 240. The bar is placed into the apparatus as shown in FIG. 2B by placing the lock plate (230) onto the locking end of the bar. In this example the locking plate has a through hole which is aligned with the collar and a portion of the lock end of the bar (140) passes through the lock plate. In this manner the bar is correctly aligned with the collar and the quenchant discharge apparatus.

As shown in FIGS. 2A and 2B, the quenchant discharge apparatus (300) preferably comprises a quenchant nozzle (310). The quenchant discharge apparatus may further comprise a conduit (320) fluidly connected between the quenchant nozzle and a quenchant storage source (not shown). Within the conduit between the quenchant nozzle and the quenchant storage source may be a valve (330). The valve may have a valve position selected from the group consisting of open, closed, and partially open. Common examples of valves include ball valves, butterfly valves, check valves, globe valves, gate valves, solenoid valves and plug valves When the valve is in the open or partially open position, an amount of quenchant is allowed to pass from the quenchant storage source through the conduit to be discharged from the quenchant nozzle.

There may also be a filter, in particular a depth filter, (315) to equalize the pressure within the nozzle.

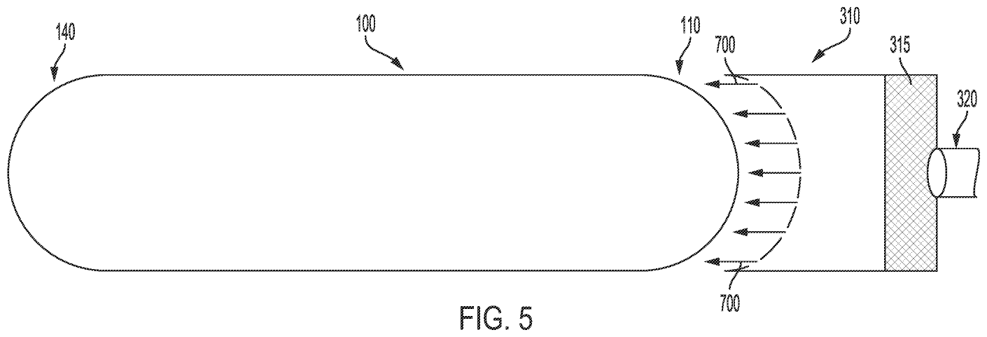

A preferred nozzle quenchant discharge is disclosed in FIG. 5 which shows nozzle (310) having multiple sprays as indicated by the arrows. In this example, the nozzle is configured so that each spray outlet is the same distance from the bar surface to keep the quench more uniform at each point on the surface.

Another alternative is to vary the hole size of each outlet and/or distance from the surface so that there is a slight pressure differential which adds additional pressure to force the quenchant even more from the center of the tip more.

In another embodiment, the apparatus is operated so that the discharge of the quenchant is with the force of gravity so that gravity is pulling the quenchant away from the surface and into the shield. One of ordinary skill would know that a drain would be needed in the shield to remove the water after it has flowed off of the tip of the bar.

Preferably, the quenchant will be discharged from the quenchant nozzle such that the metal composition of the bar is cooled at a cooling rate equal to or greater than the metal composition's critical cooling rate as discussed in International Patent Publication No. WO/2019/157075, the teachings of which are incorporated by reference herein in their entirety.

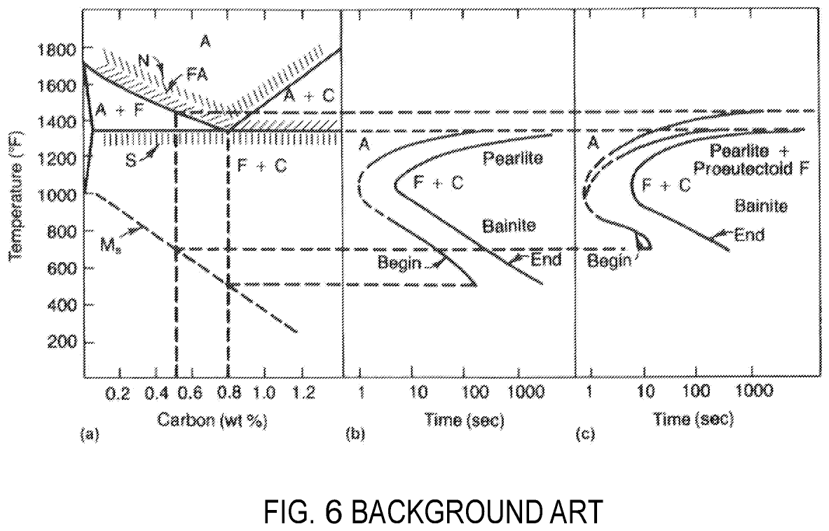

The critical cooling rate is established by the inflection point on the TTT diagram of the material making up the part and the part's starting surface temperature. In the case the modified Jominy bar's, starting surface temperature. It can be seen from the TTT diagram of FIG. 6, which is applicable only to the material from which it was derived, that this material's inflection point occurs in approximately 1 sec at 1100° F. (593° C.). Assuming the starting surface temperature of the part is 1600° F. (871° C.), the required cooling rate to keep the surface of the part out of the intermediate phase (i.e. the critical cooling rate) is the surface temperature at the start (Ts) of the quench less the temperature at the inflection point (Ti) divided by the time of the inflection point (ti). In this case, Ts=871, Ti=593 and ti=1 yielding a critical cooling rate of at least: [871−593]/1=278° C. per sec in order to miss the nose of the curve.

It is therefore important when one wants to establish compressive stresses to cool the surface of the part at a rate equal to or greater than the part's critical cooling rate for at least the amount of time corresponding to the inflection point (ti).

It is important to understand what happens in this one second. In many instances, it takes many seconds, sometimes 40 seconds to a minute, or more, to move the hot part from the heat source to the quench tank and begin the quenching cycle. However, there is an “air quench” occurring in the 40 seconds of transfer. But more importantly, if the rapid quench is not started before the time of the inflection point, non-uniformity of the quench begins to affect the layers below the surface.

While it is preferable that the critical cooling rate begin at a time less than the time associated with the inflection point (ti) (i.e. the quenchant's initial contact with the part surface occurs at a time less than the time associated with the inflection point (ti); it is most preferable to have no air quench, or no cooling, between the moment the heating stops and the moment the part is contacted with the quenchant creating an instantaneous quench.

One way to accomplish this is to inductively heat the part and turn on the quench immediately or simultaneously with or prior to turning off the induction unit. The use of the current apparatus immediately removes the inductively heated modified Jominy bar from the heating tool and drops it into the apparatus. The quench starts immediately upon the locking of the handle.

The part surface critical cooling rate may be a rate of at least 278° C. per second.

The part surface critical cooling rate may be a rate of at least 300° C. per second.

The part's surface cooling rate of at least 300° C. per second is established by many parameters known to those of ordinary skill. For example, the liquid quenchant itself will have a specific gravity, a temperature and a rate of thermal conductivity, the part surface will have thermal conductivity as well as the heat flow though the part. The temperature differential between the part surface and the liquid quenchant also plays a large role. The rate of liquid quenchant renewal at the part surface, which in some instances could be the mass velocity over the part, also plays a role. It is believed that the part surface cooling rate need only to be maintained for less than a second, or even a half a second. But the cooling rate of at least 300° C. per second is believed to be the minimum rate at the initial moment of contact of the liquid quenchant with the part surface. It is believed that one of the reasons for this high rate of cooling is to form the Martensite in the grains below the surface layer.

Additionally, a method for determining the inherent ultimate hardenability and available compressive stresses of a metal composition having an Austenitizing temperature is disclosed. This method first involves heating a bar (100) having a non-flat, preferably hemispherical, tip (110) to a through temperature equal to or greater than the Austenitizing temperature of the metal composition of the bar according to any one of the methods disclosed herein. Next at least a portion of the non-flat, hemispherical tip, of the bar may be exposed through a bar receptacle (200). The portion of the non-flat hemispherical tip of the bar exposed is subjected to a quenchant at a pressure greater than a boiling pressure of the quenchant at the through temperature of the bar (unless specifically testing an “air quench” alloy material) and at a quenchant flow rate for a defined time released from a quenchant nozzle (310). After quenching the bar can be analyzed for at least one property, examples of which are ultimate hardenability, compressive stresses, tensile stresses, crystal structure, grain size, hardness, and combinations thereof.

### EXPERIMENTAL

Experiments were conducted with two different nozzles on the same modified bar made from the same alloy (1045 Steel). The bar was hemispherical on both ends.

Both bars were inductively heated to above the Austentizing temperature. They were both transferred to the apparatus which upon addition of the bar became the system.

Each bar was quenched with 60 gpm (227.1 Lpm) of water at the same ambient temperature at 60 psig (4.137 barg).

One of the hemispherical bars were quenched using a single spray. The other bar was quenched using a multiple spray nozzle as shown in FIG. 5.

The quenched tip of the multiple spray test bar was analyzed under X-RAY DIFFRACTION (XRD) in compliance with EN 15305: Non-destructive Testing-Test Method for Residual Stress Analysis by X-ray Diffraction, 2008; ASTM E915-16: Standard Test Method for Verifying the Alignment of X-Ray Diffraction Instrumentation for Residual Stress Measurement. Exception: the inventor used an epoxy mounted powder sample for durability, SAE HS-784: Residual Stress Measurement by X-Ray Diffraction, 2003. Exception: The inventor used Modified-Chi detector geometry instead of Chi/Psi geometry described by the standard and follows the calculation guidelines set forth by EN 15305., and ASTM E2860-12: Standard Test Method for Residual Stress Measurement by X-Ray Diffraction for Bearing Steels. Exception that the calculation and collimator size guidelines were done according to those set forth by EN 15305.

| | 0° | | 45° | | 90° | |
|---|---|---|---|---|---|---|
| Depth | Stress | Dev. | Stress | Dev. | Stress | Dev. |
| [μm] | [MPa] | [MPa] | [MPa] | [MPa] | [MPa] | [MPa] |
| 0 | −206 | 24 | −173 | 20 | −203 | 20 |
| 34 | −325 | 22 | −316 | 25 | −240 | 12 |
| 130 | −442 | 18 | −450 | 23 | −426 | 14 |
| 212 | −403 | 15 | −411 | 17 | −397 | 19 |
| 270 | −425 | 24 | −434 | 19 | −428 | 20 |
| 350 | −422 | 15 | −425 | 15 | −410 | 17 |
| 440 | −401 | 12 | −410 | 24 | −387 | 20 |
| 520 | −340 | 17 | −368 | 19 | −333 | 21 |
| 631 | −256 | 22 | −308 | 17 | −272 | 20 |
| 719 | −269 | 25 | −299 | 17 | −273 | 24 |
| 819 | −215 | 13 | −226 | 15 | −209 | 18 |
| 893 | −169 | 17 | −208 | 22 | −153 | 20 |
| 1010 | −120 | 16 | −155 | 23 | −143 | 14 |
| 1100 | −113 | 13 | −144 | 20 | −108 | 23 |
| 1220 | −4 | 15 | −64 | 13 | −47 | 17 |
| 1366 | 8 | 12 | −43 | 14 | −6 | 19 |
| 1505 | 73 | 16 | −33 | 11 | −7 | 15 |
| 1644 | 57 | 22 | 18 | 14 | 41 | 17 |
| 1789 | 99 | 22 | 12 | 19 | 63 | 14 |

| | Max | Min | | |
|---|---|---|---|---|
| Depth | Stress | Stress | Dev | Max Stress dir. |
| [μm] | [MPa] | [MPa] | [MPa] | [°] |
| 0 | −166 | −226 | 18 | 20 |
| 34 | −301 | −326 | 19 | 71 |
| 130 | −448 | −478 | 22 | 82 |
| 212 | −345 | −470 | 22 | −25 |
| 270 | −428 | −441 | 17 | −26 |
| 350 | −365 | −497 | 22 | −79 |
| 440 | −361 | −443 | 17 | −53 |
| 520 | −309 | −409 | 22 | −55 |
| 631 | −202 | −331 | 17 | −28 |
| 719 | −271 | −300 | 21 | −24 |
| 819 | −226 | −245 | 15 | −85 |
| 893 | −86 | −239 | 20 | −40 |
| 1010 | −114 | −162 | 15 | −7 |
| 1100 | −69 | −166 | 20 | −34 |
| 1220 | 19 | −94 | 15 | −35 |
| 1366 | 55 | −48 | 15 | −15 |
| 1505 | 80 | −32 | 14 | −12 |
| 1644 | 79 | 24 | 15 | −35 |
| 1789 | 82 | 42 | 16 | −12 |

As the above tables demonstrate the modified Jominy bar was able to establish a stress profile based upon very controlled known conditions. These residual compressive stresses, and their distances based upon instantaneous intensive quenching with a high quenchant renewal rate can then be used to characterize the metal alloy of the bar. This characterization is then coupled with finite element analysis regarding compressive and tensile stresses established in the few microseconds of the quench and that happen at the speed of sound. These now known values which have been empirically attained can be used to predict net shape change during the quench and eliminate post carburization treatments. Once the net shape change during the quench is modeled, the green part can be made knowing that it will distort into the proper shape.

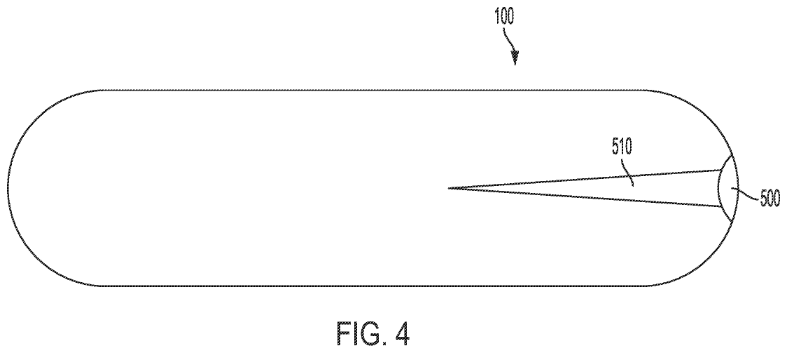

The advantage of the multiple spray versus the single spray can be seen in FIG. 4. One analysis is to cut the bar along its longitudinal axis and determine stresses and hardness from the surface to the center of the bar. Analysis showed that the point of impact (500), (approximately 0.2 mm) was visibly present as a dot. It is believed that the tip was initially hard but that the remaining heat flow flipped that part from compressive stress to residual stresses. However, behind the tip was a plume (510) which exhibited the same high residual compressive stresses and hardness that shrank to the longitudinal axis. This information is again useful in establishing the quenchant volumetric flow rates and pressures.

This invention addresses the flaws of traditional Jominy end-quench tests discussed above, including cooling inconsistencies in the flat area of the bar (100) impacted by the quenchant. The inconsistencies in the area of the bar is addressed by exposing a consistent portion of the hemispherical tip of the bar to quenchant. The time spent transferring the bar to the bar receptacle is addressed by heating the bar in the same system with little to no time involved in the physical transfer of the bar from the heat source to the tester which reduces the amount of time between the end of the heating cycle and the beginning of the liquid quenching cycle.

1. A system for conducting a modified Jominy end-quench test of a metal composition having an Austenitizing temperature and a critical cooling rate comprising:
a bar comprising the metal composition, said bar having a hemispherical tip and a bar length;
a bar receptacle comprising a rigid collar with a top surface, a bottom surface opposite the top surface, a through hole from the top surface through the bottom surface, and a brace;
a quenchant;
a quenchant discharge apparatus comprising a quenchant nozzle; and
wherein a portion of the hemispherical tip of the bar passes through the through hole to form an exposed portion of the hemispherical tip of the bar which is located between the bar receptacle and the quenchant nozzle with a gap existing between the quenchant nozzle and the hemispherical tip.

2. The system of claim 1, wherein the quenchant nozzle is configured to discharge the quenchant to cool at least the exposed portion of the hemispherical tip of the bar at a cooling rate equal to or greater than a critical cooling rate of the metal composition of the bar.

3. The system of of claim 1, further comprising a shield attached to the bar receptacle.

4. The system of claim 1, further comprising a reinforcement, connecting the bar receptacle and the shield.

5. The system of claim 1, wherein the gap between the quenchant nozzle and the hemispherical tip is between 5 millimeter and 5 centimeters.

6. The system of claim 1, wherein the gap between the quenchant nozzle and the hemispherical tip is between 5 millimeter and 2.5 centimeters.

7. The system of claim 1, wherein the system is configured to cool the exposed portion of the hemispherical tip without film boiling.

8. A method using the system of claim 1 to determine at least one property of the metal composition, said method comprising:
A. heating at least a surface of the bar at the exposed portion of the hemispherical tip of the bar to a temperature greater than or equal to the Austenitizing temperature;
B. passing at least a portion of the hemispherical tip of the bar through the through hole of the bar receptacle;
C. shielding all but the exposed portion of the hemispherical tip of the bar to keep the quenchant from contacting any surface of the bar other than the exposed portion of the hemispherical tip of the bar;
D. contacting the exposed portion of the hemispherical tip of the bar with the quenchant; and
E. analyzing the bar for the at least one property.

9. The method of claim 8, wherein the exposed portion of the hemispherical tip of the bar is contacted with the quenchant at a pressure greater than a boiling pressure of the quenchant at the through temperature of the bar and at a quenchant flow renewal rate for a time.

10. The method of claim 8, wherein the at least one property is selected from a group consisting of ultimate hardenability, compressive stresses, tensile stresses, crystal structure, grain size, hardness, and combinations thereof.

11. The method of claim 8, wherein the metal composition is in the shape of a bar is heated to at least the Austenitizing temperature using induction heating.

12. The method of claim 8, wherein the hemispherical tip is cooled by the quenchant at an initial cooling rate equal to or greater than the critical cooling rate of the metal composition.

13. The method of claim 8, wherein the quenchant exiting the quenchant nozzle is located between 5 millimeters and 5 centimeters from the hemispherical tip.

14. The method of claim 8, wherein the quenchant exiting the quenchant nozzle is located between 5 millimeters and 2.5 centimeters from the hemispherical tip.

15. The method of claim 8, wherein the method further comprises analyzing compressive surface stresses along the bar length.

16. The method of claim 8, wherein the method further comprises determining the ultimate hardenability of the metal composition of the bar through measuring hardness at a plurality of depths.
